(19) United States
(12) Patent Application Publication
(10) Pub. No.: US 2008/0188713 A1
(43) Pub. Date: Aug. 7, 2008

(54) Deformable Flexible Tube to be Used in Endoscopes

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/776,863

(22) Filed: Jul. 12, 2007

(30) Foreign Application Priority Data
Feb. 7, 2007 (TW) 096202388

(51) Int. Cl. F16L 11/00; A61B 1/005; A61M 16/00

(52) U.S. Cl. 600/139; 128/207.14; 138/118; 600/109

(57) ABSTRACT

A flexible tube to be used with a hollow tube is disclosed. The flexible tube comprises a flexible body which comprises a front end, a rear end, and an elastic segment located between the front end and the rear end. The elastic segment is capable of changing its shape on the basis of the variation of its position relative to the hollow tube so as to change the orientation of the front end.

### BACKGROUND OF THE INVENTION

1. Field of the Invention

The present invention relates to a flexible tube which can be used in a hollow tube, such as an endotracheal tube. More particularly, the invention relates to a flexible tube capable of changing its shape on the basis of the variation of its position relative to the hollow tube; said flexible tube may be applied to various endoscopes.

2. Description of the Related Art

Endotracheal intubation is a critical means for maintaining the breathing function of a patient under general anaesthesia. In most cases, to prevent the occurrence of hypoxia, anaesthetists have to complete the intubation by inserting an endotracheal tube into the patient's trachea in a very short period of time to provide oxygen thereinto promptly. Thus, it is vital that anaesthetists perform the intubation efficiently.

However, due to the structural differences of the upper airways among patients of different ages or sizes, the patients' tracheas sometimes cannot be easily located. Thus, in practice, anaesthetists always have auxiliary tools at hand to facilitate the intubation process. In some cases, laryngoscopes are adapted to depress the patient's tongue base, and a tracheoscope with a photographic device may then be inserted into the upper airway to observe the location of the trachea. This visual location of the trachea with the tracheoscope allows the endotracheal tube, which is sleeved on the tracheoscope in advance, to be delivered into the patient's trachea as soon as the trachea is located. In these cases, the time wasted by random, empirical searching for the trachea can be largely reduced, and injury to tissues of the patient's upper airway resulting from intubation may be avoided. For further introduction of these types of products, please refer to http://www.yuanyu.tw/medical/Product_Type1.asp?Product_Type (last visit: 21 Jun. 2007).

Even though the usage of a tracheoscope with an endotracheal tube may facilitate the intubation process, there are still some inconveniences in practice. For example, most users encounter the problem that the position of the trachea cannot be determined due to the limited fixed shooting angle of the photographic device. Although some tracheoscopes provide certain angle-controlling mechanisms, all of the currently available mechanisms must be operated with two hands, and most of those angle-controlling mechanisms are rather complicated. Thus, when these tracheoscopes are applied in more difficult cases, such as patients with shorter necks or thicker tongues, these instruments may endanger the patients due to the inconvenience and time involved in operating them. Particularly noteworthy is that even though these instruments are of great help in tracheal positioning and intubation, they are not common in hospitals with smaller budgets due to their intimidating, unaffordable prices.

In view of the fact that efficient intubation is critical for the survival of a patient, there is a need to provide a device capable of changing its shape when applied to the upper airways of different patients; furthermore, the device must be affordable even for smaller hospitals and provide simple, quick operation.

### SUMMARY OF THE INVENTION

It is therefore an object of the present invention to provide a flexible tube which may be used with a hollow tube, such as an endotracheal tube; the flexible body of said flexible tube has an elastic segment for causing deformation.

It is another object of the present invention to provide a flexible tube disposed with an elastic piece; when used with a hollow tube, the elastic piece may change its shape on the basis of the variation of its position relative to the hollow tube so as to change the shape of the flexible tube.

It is still another object of the present invention to provide an elastic piece to be secured to an ordinary flexible tube. When the flexible tube is used together with a hollow tube, the elastic piece may deform on the basis of the variation of its position relative to the hollow tube so as to adjust the angle of the front end of the flexible tube protruding from the hollow tube.

To achieve the above-mentioned purposes, the present invention provides a flexible tube which may be used with a hollow tube such as an endotracheal tube. The flexible tube comprises a flexible body which comprises: a front end; a rear end; and an elastic segment located between the front end and the rear end, wherein the elastic segment is capable of changing its shape on the basis of the variation of its position relative to the hollow tube so as to change the orientation of the front end. It is appreciated that in all of the above aspects of the invention, the elastic segment may be fabricated with shape-memorizing material, such as nickel-titanium alloy. In addition, the elastic segment may also be fabricated with, but not limited to, flexible material, such as pre-shaped rubber. In use, the position of the flexible tube relative to the hollow tube may be adjusted by a user in order to create bends with different curvatures. Accordingly, the flexible tube is suitable for various kinds of upper airways, and it is capable of facilitating the intubation process in different patients.

To further improve the convenience of the flexible tube in use, an illumination unit, such as an LED light bulb, may be installed at the front end of the flexible tube, said illumination unit being electrically connected to a power supply installed at the rear end of the flexible tube. With this installation, users may turn on the illumination unit to light up the deeper part of the patient's upper airway when the flexible tube is inserted therein. In addition, an image-capturing unit may also be installed at the front end of the flexible tube, thus making it a tracheoscope capable of changing its shooting angle. In brief, the flexible tube of the present invention may be fitted with different modules to satisfy users' needs.

Moreover, the present invention also provides a flexible tube which may be used with a hollow tube, said flexible tube comprising a flexible body and an elastic piece secured to the flexible body, wherein the elastic piece is capable of changing its shape on the basis of the variation of its position relative to the hollow tube so as to change the shape of the flexible body. Similarly, this kind of flexible tube, if necessary, may also be fitted with an illumination unit, a power supply, and/or an image-capturing unit to satisfy users' needs.

Furthermore, the present invention also provides an elastic piece to be secured to a flexible tube inserted into a hollow tube. The elastic piece is capable of changing its shape on the basis of the variation of its position relative to the hollow tube so as to cause the deformation of the flexible tube accordingly. When the elastic piece is secured to the flexible body of an ordinary tracheoscope, users may perform the photographic task from different angles, simply by adjusting the relative positions of the elastic piece and the hollow tube. In addition, the elastic piece may be made of shape-memorizing material, such as nickel-titanium alloy; it may also be made of, but not limited to, flexible material, such as pre-shaped rubber. Furthermore, the elastic piece may be fabricated into various forms, such as a spring, an elastic ring, an elastic strip, etc.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustrations only, and not as a definition of the invention.

In the drawings, wherein similar reference numerals denote similar elements throughout the several views:

FIG. 1 is a perspective view of one embodiment of the flexible tube of the present invention.

FIGS. 2A to 2C illustrate other embodiments of the flexible tube of the present invention.

FIG. 3 illustrates how the elastic piece of the present invention deforms on the basis of the variation of its position relative to the endotracheal tube.

FIGS. 4 to 6 illustrate the statuses of the elastic piece of the present invention when used with an ordinary flexible tube.

### DETAILED DESCRIPTION OF THE PREFERRED EMBODIMENT

These and other features of the invention will become more apparent with a discussion of the various embodiments in reference to the associated drawings.

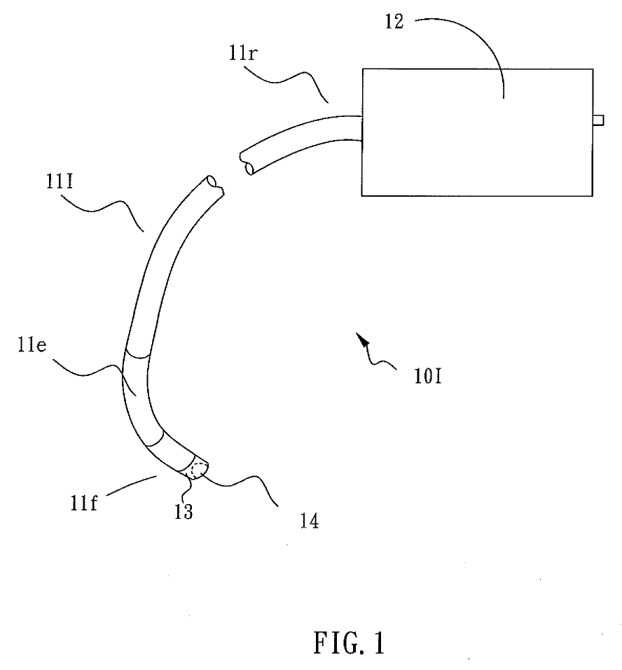

Please refer to FIG. 1 for a perspective view of one embodiment of the flexible tube 10I of the present invention. The flexible tube 10I mainly comprises a flexible body 11I, which has a front end 11f, a rear end 11r, and an elastic segment 11e; said elastic segment 11e is located between the front end 11f and the rear end 11r, and the elastic segment 11e is capable of changing its shape on the basis of the variation of its position relative to a hollow tube so as to change the orientation of the front end 11f. The elastic segment 11e may be made of shape-memorizing material, such as nickel-titanium alloy; in addition, the elastic segment 11e may also be made of, without limitation, shape-memorizing flexible material, such as pre-shaped rubber. In addition, to provide the flexible tube 10I with a lighting function, the flexible tube 10I further comprises a power supply 12 installed at the rear end 11r and an illumination unit 13 installed at the front end 11f and electrically connected to the power supply 12. Thus, when users are to insert the flexible tube 10I into the deeper part of the patient's upper airway, they may turn on the illumination unit 13 so as to illuminate the part to be observed, facilitating the observation. In the meantime, the flexible tube 10I may also be fitted with an image-capturing unit 14 and thereby become a tracheoscope. The image-capturing unit 14 is electrically connected to the power supply 12 and is capable of recording the images of the inside of the patient's upper airway; the images may be displayed on a display unit, which may be installed externally or directly on the power supply 12.

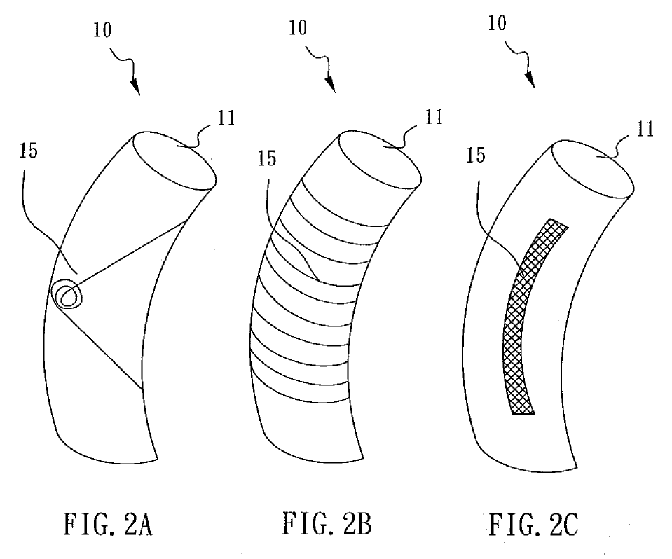

Refer now to FIGS. 2A to 2C for illustrations of other embodiments of the flexible tube 10 of the present invention. To enable an ordinary flexible body 11 of a tube (a flexible tube without an elastic segment, such as the body of an ordinary tracheoscope) to change its angle, the present invention provides an elastic piece 15 to be secured to the ordinary flexible body 11 of a tube, providing it with a shape-varying characteristic. For example, consider an ordinary tracheoscope. After the flexible body 11, which is disposed with the elastic piece 15, is inserted into a hollow tracheoscope, the elastic piece 15 is capable of changing its shape on the basis of the variation of its position relative to the tracheoscope, consequently changing the shooting angle of the image-capturing device installed at the front of the flexible body 11. As shown in FIGS. 2A to 2C, the elastic piece 15 may be a spring, an elastic ring, or an elastic strip. However, it should be noted that, although only three different forms of the elastic piece 15 are disclosed herein, the scope of this invention should also cover other derivatives having an elastic characteristic or being made of shape-memorizing material.

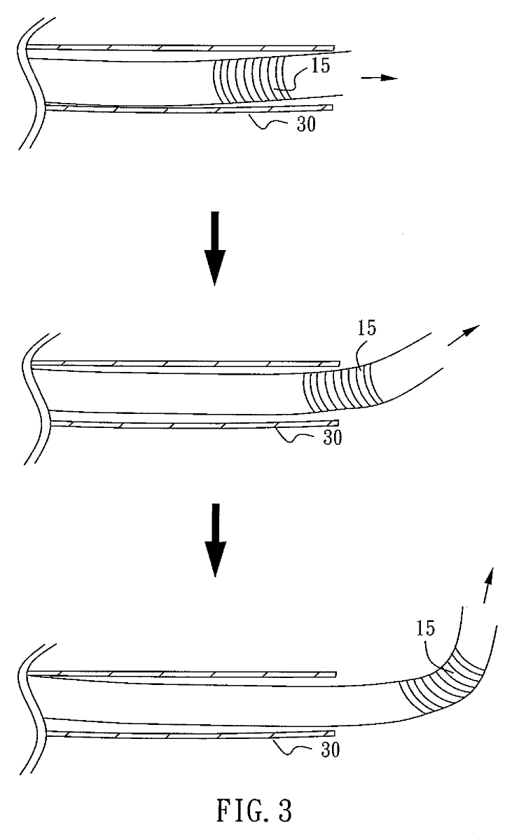

Refer now to FIG. 3 for an illustration of how the elastic piece 15 of the present invention deforms on the basis of the variation of its position relative to the endotracheal tube 30. As shown, when the elastic piece 15 is completely encompassed by the endotracheal tube 30, the elastic piece 15 may be restrained under an expanded status due to the structural limitation of the endotracheal tube 30; as the elastic piece 15 gradually emerges from the endotracheal tube 30, it may then change its shape due to the inherent resilience; once completely free of the restraint of the endotracheal tube 30, the elastic piece 15 may then return to its original shape without any limitation.

To illustrate the achievable effect provided by the elastic piece 15 used with the ordinary flexible body 11 of a tube, several figures are disclosed hereinafter.

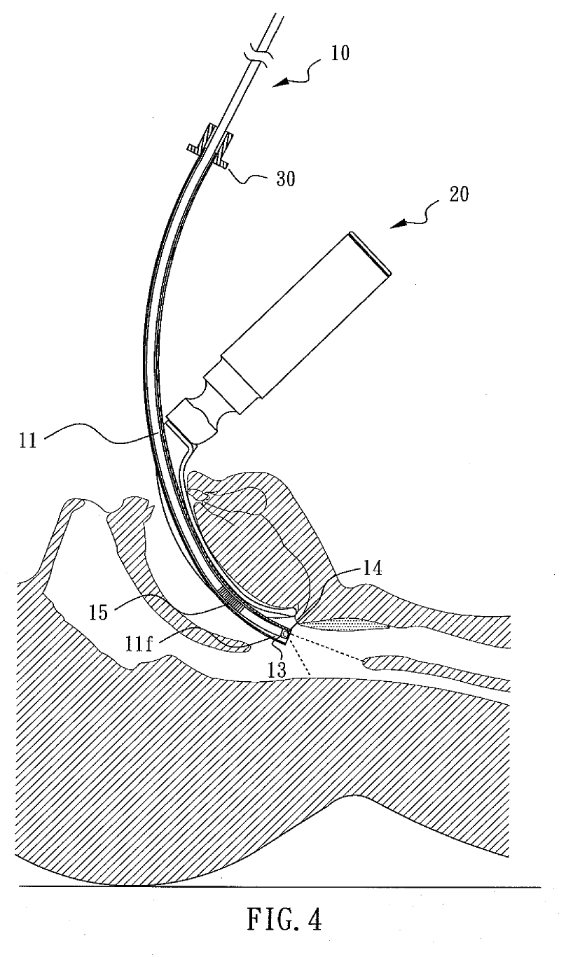

Please refer to FIG. 4 to FIG. 6, which illustrate the statuses of the elastic piece 15 of the present invention inserted in the flexible body 11 of an ordinary flexible tube 10, such as an endoscope. During the intubation process, the head of a patient is held backward so as to raise his/her chin, making the trachea more observable. Then a laryngoscope 20 is inserted into the patient's mouth to depress his/her tongue base so that the opening of the trachea may not be inhibited by epiglottis cartilage. Subsequently, the flexible tube 10, which is inserted into the endotracheal tube 30, is inserted into the patient's mouth, wherein the elastic piece 15 is secured to the flexible body 11 of the flexible tube 10. If an image-capturing unit 14 is installed at the front end 11f of the flexible tube 10, users may then observe the image of the patient's upper airway and simplify the placement of the flexible body 11 of the flexible tube 10 and the endotracheal tube 30. As shown in FIG. 4, when the elastic piece 15 is completely encompassed within the endotracheal tube 30, the elastic piece 15 is restrained under an expanded status due to the structural limitation, for example, curvature, of the endotracheal tube 30.

However, under certain circumstances, if the structure of the patient's upper airway is unfavorable for observation (in the case of a patient with a shorter neck or a thicker tongue), users cannot direct the image-capturing unit 14 to a position capable of viewing the trachea by simply pushing the endotracheal tube 30 and the flexible tube 10 (as shown in FIG. 4, where the image-capturing unit 14 may only view the esophagus). In these cases, users may fix the endotracheal tube 30 and at the same time push forward the flexible body 11 of the flexible tube 10 relative to the endotracheal tube 30 so as to vary the position of elastic piece 15 relative to the endotracheal tube 30.

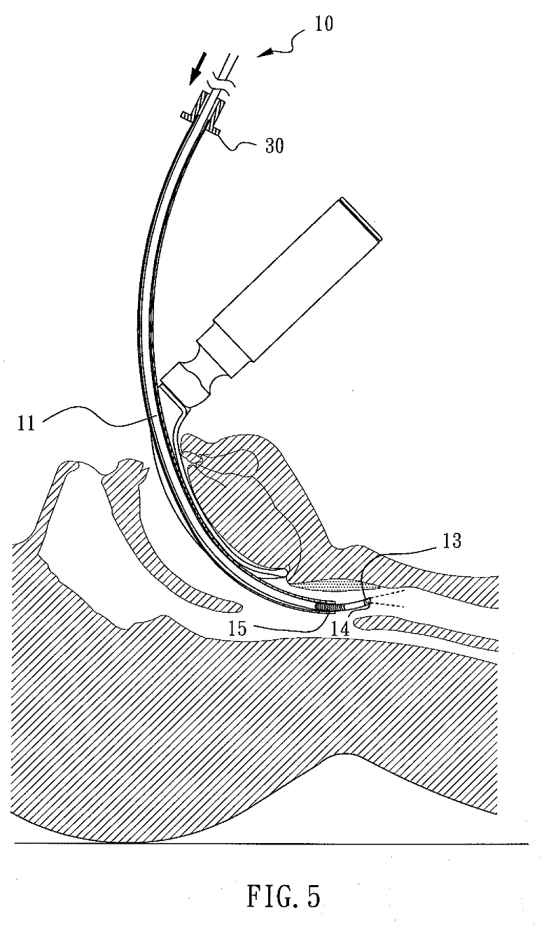

As shown in FIG. 5, when the elastic piece 15 is pushed out of the endotracheal tube 30 partially, it is no longer restrained by the endotracheal tube 30 and is capable of representing a different curvature due to a deformation caused by its inherent resilience; meanwhile, the shooting angle of the image-capturing unit 14 installed at the front end 11f of the flexible body 11 may be changed to a favorable status capable of locating the trachea and enabling users to observe the trachea.

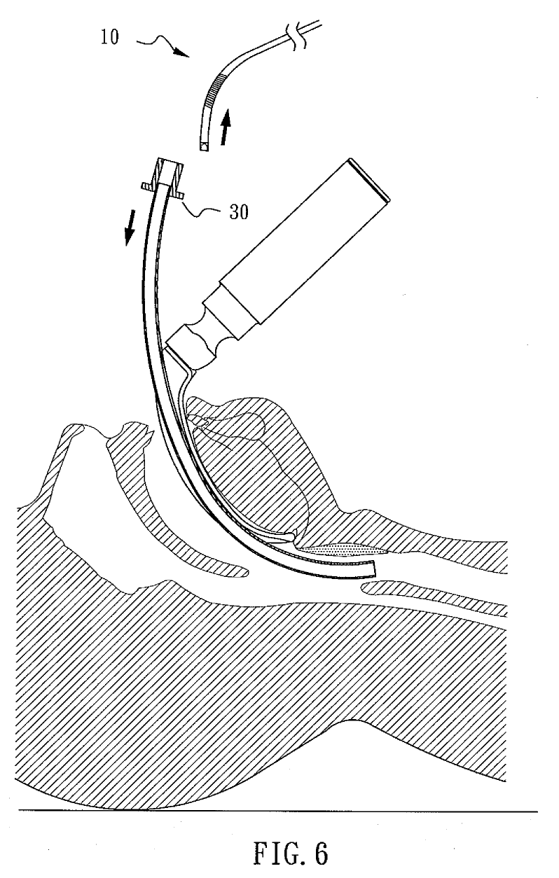

Once the trachea is found, users may then fix the flexible tube 10 and push the endotracheal tube 30 along the flexible body 11 of the flexible tube 10 into the patient's trachea. As shown in FIG. 6, users may then retract the flexible tube 10 and connect the endotracheal tube 30 to an oxygen supplying device to complete the intubation.

It will be understood that many other modifications can be made to the various disclosed embodiments without departing from the spirit and scope of the invention. For these reasons, the above description should not be construed as limiting the invention, but should be interpreted as merely exemplary of preferred embodiments. For example, users may also use the flexible tube 10I with an elastic segment 11e of the present invention to adjust the shooting angle shown in FIGS. 4 to 6.

What is claimed is:

1. A flexible tube to be used with a hollow tube, said flexible tube comprising a flexible body which comprises:
a front end;
a rear end; and
an elastic segment located between the front end and the rear end, wherein the elastic segment is capable of changing its shape on the basis of the variation of its position relative to the hollow tube so as to change the orientation of the front end.

2. The flexible tube as claimed in claim 1, wherein the elastic segment consists of shape-memorizing material.

3. The flexible tube as claimed in claim 1, further comprising:
a power supply installed at the rear end; and
an illumination unit installed at the front end and electrically connected to the power supply.

4. The flexible tube as claimed in claim 3, wherein the illumination unit is an LED light bulb.

5. The flexible tube as claimed in claim 3, further comprising an image-capturing unit for capturing an image, said image-capturing unit being installed at the front end and electrically connected to the power supply.

6. The flexible tube as claimed in claim 1, wherein the hollow tube is an endotracheal tube.

7. A flexible tube to be used with a hollow tube, said flexible tube comprising a flexible body and an elastic piece secured to the flexible body, wherein the elastic piece is capable of changing its shape on the basis of the variation of its position relative to the hollow tube so as to change the shape of the flexible body.

8. The flexible tube as claimed in claim 7, further comprising:
a power supply installed at one end; and
an illumination unit installed at the other end, said illumination unit being electrically connected to the power supply.

9. The flexible tube as claimed in claim 8, wherein the illumination unit is an LED light bulb.

10. The flexible tube as claimed in claim 8, further comprising an image-capturing unit for capturing an image, said image-capturing unit being installed at the same end as the illumination unit and electrically connected to the power supply.

11. The flexible tube as claimed in claim 7, wherein the elastic piece consists of shape-memorizing material.

12. The flexible tube as claimed in claim 7, wherein the elastic piece is a spring.

13. The flexible tube as claimed in claim 7, wherein the elastic piece is an elastic ring.

14. The flexible tube as claimed in claim 7, wherein the elastic piece is an elastic strip.

15. The flexible tube as claimed in claim 7, wherein the hollow tube is an endotracheal tube.

16. An elastic piece to be secured to a flexible tube that may be inserted into a hollow tube, wherein the elastic piece is capable of changing its shape on the basis of the variation of its position relative to the hollow tube so as to cause the deformation of the flexible tube.

17. The elastic piece as claimed in claim 16, wherein the elastic piece consists of shape-memorizing material.

18. The elastic piece as claimed in claim 16, wherein the elastic piece is a spring.

19. The elastic piece as claimed in claim 16, wherein the elastic piece is an elastic ring.

20. The elastic piece as claimed in claim 16, wherein the elastic piece is an elastic strip.
